(19) United States
(12) Patent Application Publication
(10) Pub. No.: US 2007/0072190 A1
(43) Pub. Date: Mar. 29, 2007

(54) System and method for universal identification of biological humans

(76) Inventor: Abhinav Aggarwal, Chapel Hill, NC (US)

Correspondence Address: DR. ABHINAV AGGARWAL, 100 SAINT AYERS WAY, CHAPEL HILL, NC, 27517, US

(21) Appl. No.: 10/735,333

(22) Filed: Dec. 12, 2003

Related U.S. Application Data
(60) Provisional application No. 60/433,226, filed on Dec. 16, 2002.

(51) Int. Cl. C12Q 1/68

(52) U.S. Cl. 435/6

(57) ABSTRACT

A system and method for uniquely identifying each human being and generate a Universal Identification Number (UIN) to aid tracking. UIN database comprises of public, health, and confidential information. The number issued for display on UIN Identity Card and used for accessing data on-line protects privacy of the individual by hiding sensitive informafion. Medical emergency data is provided to authorized hospitals and medical practitioners for advance preparedness to save lives. UIN database is accessible to law enforcement agencies, when authorized under specific circumstances. UIN prevents identity fraud by protecting unique identity of every human and helps in tracing lost or wanted people. UIN fulfills a pending need for technological system to eliminate child abduction, identity fraud, credit card theft, and help in saving lives of people. Future UIN applications relate to biosensor integrated device interacting with radio signals for monitoring and rescuing patients.

### BACKGROUND OF THE INVENTION

Field of the Invention: Human Identification.

Present invention relates to a system and method for uniquely identifying biological humans.

### BACKGROUND ART

Currently available systems don't specifically address the issues of a globally available identification mechanism that covers each and every individual on the planet.

Unique Identification of biological humans is of critical importance. At the same time, data related to the biological human that uniquely identifies, is of a critical nature and must be available upon requirement in the emergency situations to save human life. For example, a particular patient's blood type shall reveal an important data that can be crucial to saving time for an immediate medical treatment. Also, prior knowledge about allergies can prevent a wrong drug from being administered. In addition, complete confidential data that can be of vital importance for law enforcement agencies must be available at all times for verification and prevention of identity frauds.

At present, there is no system and method that uniquely identifies a human being on a global basis. Several national initiatives in this regard hold data about individuals in an isolated and disjointed manner. There may or may not be a cross-reference between several identities that a human being may hold. For instance, a Social Security Card may not reference to a driving license number. Also, data pertaining to several numbering schemes can be tapped and used by the fraudulent persons for financial frauds or criminal purposes. If someone is collecting data about a particular person even from the discarded mail, it can be possible for them to figure out a lot of personal information that is of a confidential nature. Subsequently, this information can be used with the intent of fraud. The UIN system is specifically designed to address these issues and prevent attempts at fraud.

A major problem with all the existing identification systems is that they are not global in nature, but specific only to the organization which develops and implements that particular arrangement or scheme. A particular country may use one identification scheme and an adjoining country may use a completely incompatible scheme. As a result, even though the scheme may properly identify the human within the territory of a specific nation, it may provide no usable identification when the human crosses the boundaries and visits another country. With the globalization taking place at a faster pace and the likely merging of nations, like European Union, UIN addresses the need for a technology based global human identification and tracking system.

Another significant problem with the prior art schemes is that they do not ensure confidentiality and privacy of data about the individual concerned. For example, if a person's social security number is used as the identifier for medical insurance coverage and the medical insurance policy number becomes the SSN suffixed by a zero or some other digit, all correspondence from the medical insurance company for that person will use the same identifier. As a result, the confidential data about the person is prone to be exposed and misused.

The prior art identifying arrangements and schemes may also permit tampering. For example, a person may provide the forged documents or a wrong social security number or name, while visiting another country (if they are merely seeking it for sake of record keeping). Since there is no existing arrangement of sharing data across the nations, this potential of allowance for error can result in duplicity with other identity belonging to another person having that true number or name or document.

There is no existing arrangement with any prior art identification scheme to uniquely identify a human being on a global basis, protect the privacy with an optional provision of preserving a DNA sample. Thus, UIN is a unique system and method that overcomes these shortcomings of prior art schemes.

A system and method for identifying biological humans, that overcomes the above-stated problems and yet retains the confidentiality, is not just desired but essentially required to prevent child abduction, save lives, eliminate identity frauds and eradicate terrorism from the soil of this planet.

### SUMMARY OF THE INVENTION

The present invention is a system and method for uniquely identifying and tracking biological humans, whereby each human being is associated with a universally unique identifier, the UIN (Universal Identification Number). The information associated about the individual that can be referenced with the UIN would be available upon request to authorized medical and law enforcement agencies to deal with emergency situations, saving life of the UIN holder.

Various levels of access to information that can be made available shall fall under three different domains: public, health and confidential.

At each level of access, a different authorization code is required. The authorized medical or law enforcement agency must have access to this code, to get the relevant information about the UIN holder.

The UIN card, that is issued to the UIN holder, shall contain limited information about the contact details of the individual human being and the emergency medical data. The UIN card would not contain any information that is of a confidential nature. This is to protect privacy of the individual, in case the card is lost by them and falls into the wrong hands.

The information in public and medical domains shall be stored on-line (the computer database) and made available by calling the UIN providing agency or obtained on-line upon submitting the required access and authorization codes.

Further features and advantages of the present invention over the prior art will become evident from the detailed description of the invention which follows, when considered with the attached Calculations and Algorithm details.

### BRIEF DESCRIPTION OF THE CALCULATION SHEETS AND ALGORITHMS ENCLOSED AS ATTACHMENTS

Attachment 1 details the Worksheet for calculation of Computer Generated Code Length.

Attachment 2 details the Algorithm and pseudo-code for issue of the UIN Association date.

### ADVANTAGES OF THE INVENTION

Many advantages are realized using this system and method. The invention provides a system and method for uniquely identifying all the human beings on this planet and the scheme can be universally implemented. Implementation of the method does not require the development of a specific coding scheme by a particular end user, since the unique identifier (UIN) is issued globally by a central coordinating agency. To utilize the system and method of the invention, the end user need not obtain UIN from this coordinating agency, but through the service provider network. The end user needs to only obtain the necessary UIN Card and have access to appropriate information as and when required. The end-user can also request to update or modify the information associated with the UIN in the relevant database, by providing necessary inputs and supporting documents for verification. Identity theft is the largest growing crime in America, affecting 900,000 new victims each year. (Source: www.identitvtheft.org). “Ready or not, here it comes: Identity take-over fraud has come into its own, and promises not to go away until significant changes evolve in the manner and methods by which personal identities are collected and used. Consumers would do well to arm themselves with knowledge on how to mend the damages when victimized,” says James E. Bauer, Deputy Assistant Director, Office of Investigations, US Secret Service, Washington D.C.

Further, the identifier (UIN) associated with each individual human being can be used to not only to identify the particular individual, but also to provide other information, such as the medical and confidential data to relevant authorized medical and law-enforcement agencies.

Preventing terrorism and tracing the missing people In a post-September 11 '2001 world, there is an urgent need to uniquely identify and track each and every single individual human on the planet. If we have such a system in place, tracking individuals on a global basis is possible, cutting across boundaries of the nations. Preventing terrorism and identity frauds is a responsibility for each and every nation of the world, as much as it is for each one of us. Providing a system and method that makes a paradigm shift in the ways we operate and function globally as a society is a crying need of the hour. We learn of identity frauds that lead to embezzlements worth a fortune. Your mailbox delivers images of several lost kids. Even if you had an identity kit, that would not help much beyond providing a picture of the missing child. The stage has come when we need to make an effective use of technology to protect the lives of our children and their fortunes besides ours. A missing child can be traced when anyone reports the sight and description and data can be matched with UIN database. Furthermore, UIN controlling agency can periodically issue alerts related to all missing UIN holders, seeking the cooperation of a global fraternity as human community to assist in tracing. In the futuristic plan, those opting for a tracking service can be equipped with a device that can be issued to the UIN holder and they can be tracked within a fair range of accuracy by use of satellite and radio based navigation system.

Identity Insurance

Imagine a situation when your intended heir is traveling abroad and someone who has the relevant information about you and the family poses as your heir to amass wealth by fraud. Life insurance claims can get into wrong hands. Those who truly deserve what was actually theirs would not get it. They may not have the knowledge or resource to waste a lifetime in legal proceedings and fighting court cases. The only right way to rid yourself of such potential nightmares in your life and of those who you leave behind is UIN. It is the technology answer to some of the major problems of humanity. UIN acts like Identity Insurance.

Preventing Petty Identity Frauds like Credit Card Theft

UIN card holders shall have the potential to get protection against petty thefts when Credit Card companies would be encouraged to perform a verification check of the public domain UIN data of the individual concerned. Since a fraudulent person would not be able to modify the UIN related data, they would get caught. The UIN related data is modified only on verification of a written request and appropriate documents from the UIN holder.

It is like getting the junk food out of the sight of a kid. Our current systems are not designed to handle security. Once new systems based on UIN technology are designed and put in place, it would become increasingly difficult for any one to breach the security.

In the long term plan, UIN Card is also likely to be used as an optional credit card linkage device. It would allow the invocation of a desired credit card by the UIN holder, while simultaneously authenticating the transaction.

Protecting the Environment

Technology can go a long way to help all of us to protect the environment, create peace and integrate the globe. Imagine that someone has to apply for a visa for a foreign travel. They called the embassy and gave them the UIN Number and access code. If the embassy has an authorization code to get the relevant and authentic data, the visa can be issued even electronically. All the person hours wasted in traveling and the gasoline burnt that pollutes the environment can be saved. It is just a paradigm shift in the way the world works as of today to the ways in which the world would work as of tomorrow. Even the need for a physical embassy building in that country can be eliminated if all business can be transacted electronically. UIN simply helps with a way to achieve that. With an appropriate use of UIN technology and innovative thinking, the possibilities to protect the environment are endless.

Creation of Peace and Global Integration

UIN shall become a global vehicle to connect people and governments internationally. Just like what Internet did several years ago. This endeavor is towards creating peace, eradicate terrorism, prevent frauds, save lives and integrate the globe. It is eventually an implementation of step by step technology efforts like this that would bring peace to the world and improve the quality of life.

Futuristic and Unknown Benefits

As of today, it is just the limit of our imagination that guides our perception of the anticipated advantages. Once we get the UIN technology in place, the humanity would be able to extend the cognitive boundaries of our imagination and that would enable the shifting of paradigms. The shift in attitudes from currently fighting for survival to a futuristic collaboration for growth is the turning point in human history that would be marked by UIN. The turning of tables upside down from currently competing to futuristic cooperating and creating. It is ultimately the shift in attitudes, practices, cultures and an unleashing of the hidden creative potential of a global community of humanity that would truly bring out the real benefits of UIN.

Attachment 1: Worksheet for Calculation of Computer Generated Code Length

Existing population of the world that needs to be issued UIN=approx. 7 billion

Existing human birth rate of the world=6.5 births per second

Human births in the world (highest rate data, with margins)=10 per second

Human births in the world (highest rate data, with margins)=10×60 per minute Human births in the world (highest rate data, with margins)=10×60×60 per hour

$$\begin{matrix}\begin{matrix}Human births in the world \\ ( highest rate data , with margins )\end{matrix} = 10 \times 60 \times 60 \times 24 per day \\ = 864000 births per day\end{matrix}$$

Assuming that every person on the planet was given the same first name (say AAA),
And considering that we use A to Z and 2 to 9 (excluding 0 and 1 to avoid confusion with 0 and 1) that totals to 26+7=34 alphanumeric characters such that each C (Computer generated Alphanumeric Code) holds the 34 possible options.
CC=34×34=1156
CCC=34×34×34=11256×34=39304
CCCC=34×34×34×34=39304×34=1336336
Since 1336336 exceeds 864000, an alphanumeric length of 4 characters (CCCC) is sufficient to cover all human births in the world, even if everyone was given the same first name, on any particular day. If this particular day is the UIN issue date, that date would be used as the association date.
To cover the existing 7 billion population of the world, if a particular first name can't be associated with any computer-generated code within the CCCC length limit, the immediately preceding date shall be used as the association date. See the algorithm and pseudo-code sheets for ‘Association date Algorithm.’
Attachment 2: Algorithm for UIN Association Date
UIN Issue: pseudo-code for Association Date Algorithm
Example Case: Issue of UIN for a person named Joe on 2002-11-06.
1. Pick current date YYYYMMDD as A (Association date).
2. Suffix YYYYMMDD (A) with NNN (JOE) such that YYYYMMDDNNN is now B
3. Suffix B with CCCC (Computer-generated Code).
4. The whole field is YYYYMMDDNNNCCCC. This is UIN_Temp.
5. If CCCC is PN2R, UIN_Temp is 20021106JOEPN2R.
6. Check if UIN_Temp is already in UIN_Issue list
7. If no, Issue UIN_Temp as UIN_Issue to Joe and add UIN_Temp (20021106JOEPN2R in this case) to UIN_Issue list
8. If yes, check for availability of another CCCC combination for B and make it UIN_Temp.
9. Go to 6. Keep repeating the loop of steps 6 through 8 till all possible combinations of CCCC are exhausted.
10. If there is no CCCC match available for Joe for current date YYYYMMDD (20021106 in this case), decrement current date by 1, such that new Association date (A) is now previous (last used) association date—1 (200021105 in this case).
11. Go to step 2. Keep repeating the loop of steps 2 through 10, till a valid UIN is found to break away from the loop at step 8 and becomes UIN_Issue for Joe.

What is claimed is:

1. A system for unique human identification and associated method and steps to capture and store data and DNA samples related to issue of Universal Identification Number (UIN) and a subsequent retrieval process for human identification and tracking involving various applications viz. medical emergencies, public domain interest profile, and confidential data by authorized agencies.

2. The method in accordance with claim 1, wherein said UIN as unique human identifier comprises of an association date in Year (four numerals), Month (two numerals) and Date (two numerals) format (YYYYMMDD). The association date is followed by up to first three characters of first name of a person and lastly followed by a computer generated four alphanumeric digits or characters. The UIN is such that is easy to recall and does not reveal any personal information of a confidential nature about the UIN holder and protects their privacy.

3. The method in accordance with claim 2, wherein the association date in the said UIN (as unique human identifier) comprises of a date that shall normally be the issue date for UIN. If the UIN issue date and associated name characters and the computer-generated alphanumeric characters for the issue date are non-available, then the association date is picked from those available from one of the previous dates in a time-horizon window, as explained in the attachments appended to this application.

4. The method in accordance with claim 3, wherein the association date for issue of the said UIN (as unique human identifier) is a date that shall normally be the issue date of the UIN, but in case of a clash with first three characters of the first name of the person for whom the UIN is under issue and non-availability of a suitable computer generated four alphanumeric characters, shall be successively previous date, searched and matched for issue of the UIN from the time-horizon window. This date shall be matched a day at a time, and the first previous date, that is closest to the UIN issue date, shall be picked.

5. The method in accordance with claim 2, wherein the first three characters of the first name of a person are the first three characters of a given name. In cases of non-availability of the first three characters of the first name, for reasons like the first name being shorter than three characters, the remaining characters of the first name shall be filled up by a computer-generated alphabetical sequence.

6. The method in accordance with claim 2, wherein the first three characters of the first name of a person are the first three characters of a given name. In cases of non-availability of the first three characters of the first name, for reasons like if the first name is not given owing to cultural or religious traditions or any other reasons, all characters of the first name shall be filled up by a computer-generated alphabetical sequence.

7. The system in accordance with claim 2 wherein the said UIN comprises of a number representing a date and alphabetic characters representing name and alphanumeric characters representing a computer generated code. This arrangement is such that UIN comprises of, that stands in order for YYYY (Year), MM (Month), DD (Date), NNN (Name), CCCC (Computer-generated Code). YYYYMMDD represents collectively the association date, followed by NNN for first name and CCCC for computer-generated code.

8. In accordance with claim 1, the data that is collected and makes the basis for issue of the UIN shall be divided in 3 sections, namely Public, Health and Confidential.

9. In accordance with claim 8, the first section of data collected refers to Personal Information under the public domain and captures the fields of first, middle and last name, each one of these can be 40 characters in length. Name is followed by sex (male or female) and the desired contact address with contact telephone number and e-mail address. The data in public domain shall be accessible at all times to those who have the UIN followed by the public-domain access code. The public domain access-code is known to the UIN holder, who can pass it on to anyone with whom they desire to share this information. The public domain access would reveal no more information than what is normally available on a personal business card.

10. In accordance with claim 8, the second section of data collected refers to Personal Information under the medical domain. This data captures various fields like date of birth (in YYYYMMDD format), blood type (Group and RH in 2 or 3 character width as the case may be), immediate medical conditions that may require attention (within 100 characters), details of up to 3 most severe known allergies (each can take up to 40 characters), name and contact details of the current attending physician (Name, telephone, pager, address - within 100 characters width). Emergency contact person (relative or friend) with contact details and a back-up emergency contact, each one of these can be at-most 100 characters in length. The data under medical domain shall be accessible at all times on-line or by call only to authorized hospitals and registered medical practitioners who have the UIN followed by the medical-domain access code. This data is made available to authorized viewers along with the public domain access data. Thus, the authorized viewers of medical domain data get all the relevant contact and emergency information that they may need to perform immediate treatment.

11. In accordance with claim 8, the third section of data collected refers to Personal Information under the confidential domain. This data contains all the information about UIN holder that is of a confidential nature and captures the fields as Country of birth with state, county, address of birth place, time of birth, all detailed addresses where the UIN holder has lived over 5 years, details of educational qualifications with institutes attended. Also, fields like complete present address with telephone number, country of residence, country of citizenship and details of all identifications issued so far and all numbers issued (viz. ITIN, SSN # etc.) and details of all passports and visas ever carried or issued. This data is periodically updated and the UIN holder has to own the responsibility to submit data for verification and update to the UIN issuing authority. The data related to educational qualifications including details of all schools attended, degrees or diplomas obtained, years of graduation, and subject of major. Professional affiliations, with all places of work and positions held, name of supervisor and address of workplace with dates worked. Professional societies, religious bodies or charitable and voluntary organization memberships and affiliations, positions held with dates since affiliated and addresses of the organizations. Details of birth marks and identification marks. In addition, DNA Samples of the UIN holder shall be obtained (optionally), referenced with UIN and stored at a secure site in a protected environment.

12. In accordance with claim 11, the Personal Information under confidential domain is not disclosed to anyone who is not authorized to access this information. The authorized law-enforcement agencies would be able to access this information only by a combination of three keys. The three keys are - public information access code given by the UIN holder, the security access key (authorization code) known only to the law enforcement agencies that is periodically changed to maintain confidentiality, and a third access code key that is specifically issued to the law enforcement officer requesting the information. To get the third access code, the law enforcement personnel seeking information will need to identify themselves. In order to access the information, they need to have their own UIN available for identification and verification of their personal data to eliminate any attempts at fraud. Records of all confidential data sought would be maintained for a specific period.

13. In accordance with claim 1, the UIN holder shall be issued a UIN card that carries UIN number, name of the UIN holder, picture of the UIN holder, and a thumb impression for unique identification. Additional information that is vital for life saving purposes is printed on inside of the card and not visible from the outside. The UIN card is a folded card with four visible surfaces that can hold data and information about the UIN holder. While two of the surfaces are covered in the inside fold, the two outer surfaces form the front and back of card.

14. In accordance with claim 1, the UIN shall, work as part of an integrated scheme and system that aims at eliminating terrorism, protecting identity, prevent frauds and is an attempt towards making this world a better place to be by Global Integration. UIN shall be subsequently supplemented by a tracking device that operates through satellite and radio signals, and the integrated system would become HITS (Human Identification and Tracking System).

15. In accordance with claim 1, a DNA sample shall be obtained from the UIN applicant and stored securely by the UIN issuing and identity protection agency in a secure environment. This DNA sample shall be released only in the case of the need to do so, upon appropriate request by a court of law under its authority and jurisdiction, in specific country that may normally be the residence nation of UIN holder, to settle matters related to identity fraud or eradication of terrorism. The DNA sample is referenced to the UIN.
